(19) United States
(12) Patent Application Publication
(10) Pub. No.: US 2005/0282830 A1
(43) Pub. Date: Dec. 22, 2005

(54) Use of dipyridamole or mopidamole for treatment and prevention of MMP-9-dependent disorders

(75) Inventors: Wolfgang Eisert, Hannover (DE); Andrew S. Weyrich, Salt Lake City, UT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/188,315

(22) Filed: Jul. 25, 2005

Related U.S. Application Data
(63) Continuation of application No. PCT/EP04/01091, filed on Feb. 6, 2004.
(60) Provisional application No. 60/445,741, filed on Feb. 7, 2003.

(51) Int. Cl. A61K 31/519

(52) U.S. Cl. 514/262.1

(57) ABSTRACT

A method of treatment of the human or non-human animal body for treating or preventing MMP-9-dependent disorders is disclosed, for example vascular syndromes, damages or diseases, atherosclerotic damages, arthritic conditions, inflammatory reactions, autoimmune reactions or proliferative diseases, which method comprises administering to a human or non-human animal body in need of such treatment an effective amount of a pharmaceutical composition containing dipyridamole, mopidamole or a pharmaceutically acceptable salt thereof, and the use of dipyridamole or mopidamole for the manufacture of a corresponding pharmaceutical composition.

### RELATED APPLICATIONS

This application is a continuation, under 35 USC 365(c), of PCT/EP2004/001091 filed on Feb. 6 2004.

### FIELD OF THE INVENTION

This invention relates to a method of treating and preventing MMP-9-dependent disorders using dipyridamole or mopidamole as active principle, and the use of dipyridamole or mopidamole for the manufacture of a corresponding pharmaceutical composition.

### BACKGROUND OF THE INVENTION

Dipyridamole {2,6-bis(diethanolamino)-4,8-dipiperidino-pyrimido[5,4-d]pyrimidine}, closely related substituted pyrimido-pyrimidines and their preparation have been described in e.g. U.S. Pat. No. 3,031,|450|. Further related substituted pyrimido-pyrimidines and their preparation have been described in e.g. GB 1,051,|218|, inter alia the compound mopidamol {2,6-bis-(diethanolamino)-4-piperidinopyrimido[5,4-d]pyrimidine}. Dipyridamole was introduced as a coronary vasodilator in the early 1960s. It is also well known having platelet aggregation inhibitor properties due to the inhibition of adenosine uptake. Subsequently, dipyridamole was shown to reduce thrombus formation in a study of arterial circulation of the brain in a rabbit model. These investigations led to its use as an antithrombotic agent; it soon became the therapy of choice for such applications as stroke prevention, maintaining the patency of coronary bypass and valve-replacement, as well as for treatment prior to coronary angioplasty.

Furthermore, the European Stroke Prevention Study 2 (ESPS-2; J Neurol Sci. 1996; 143: 1-13; Neurology 1998; 51: 17-19) proved that treatment by dipyridamole alone was as effective as low-dose aspirin in the reduction of stroke risk, and combination therapy with dipyridamole and aspirin was more than twice as effective as aspirin alone.

Dipyridamole appears to inhibit thrombosis through multiple mechanisms. Early studies showed that it inhibits the uptake of adenosine, which was found to be a potent endogenous anti-thrombotic compound. Dipyridamole was also shown to inhibit cyclic AMP phosphodiesterase, thereby increasing intracellular c-AMP.

In animal studies Dipyridamole has already been shown to be anti-atherosclerotic. The findings showed a combination of reduced fatty streaks as well as reduced thickening of the intima. For the past this has falsely been attributed to Dipyridamole's antiplatelet effect. The release of platelet derived growth factor (PDGF) from agitated or aggregating platelets was long thought to be the only reason for proliferation of smooth muscle cells, intima thickening and also the development of stenosis. However the modern intravascular interventions such as balloon angioplasty or placement of a metallic stent only present prothrombotic surfaces over a period of approximately one month, i.e platelet aggregation and subsequent release of PDGF is only of significance for a limited time. Proliferation and development of restenosis however develops over a much longer period of time. This indicates that other factors must be contributing significantly to the process of intima thickening, development of plaques and restenosis.

By laboratory models reflecting the complex physiology of the blood vessel it could be shown that the vasculature is not a passive conduit, but interacts profoundly with the blood through an intricate system of checks and balances to protect its integrity after vascular accident. Therefore the endothelium produces prostacyclin, a potent inhibitor of aggregation. The normal endothelium is not thrombogenic and prevents the attachment of platelets. Various stimulants precipitate the release of endothelium-derived relaxing factor (EDRF), which inhibits platelet adhesion and aggregation. At the same time, intracellular increase in cGMP was shown to be responsible for relaxation of smooth muscle cells following administration of nitro compounds. Thus the endothelium can inhibit thrombus formation by two separate mechanisms, one mediated by prostacyclin and c-AMP, and the other by EDRF and c-GMP. Dipyridamole appears to enhance both of these antithrombotic mechanisms of the vessel wall, in addition to its adenosine-sparing effects. It stimulates prostacyclin production by increasing intracellular levels of cAMP, and it enhances the strongly anti-thrombotic nitric oxide system by increasing cGMP.

Dipyridamole also has antioxidant properties (Free Radic. Biol. Med. 1995; 18: 239-247) that may contribute to its antithrombotic effect. When oxidized, low density lipoproteins become recognized by the scavenger receptor on macrophages, which is assumed to be the necessary step in the development of atherosclerosis (Ann. Rev. Med. 1992; 43: 219-25).

The inhibition of free radical formation by dipyridamole has been found to inhibit fibrinogenesis in experimental liver fibrosis (Hepatology 1996; 24: 855-864) and to suppress oxygen radicals and proteinuria in experimental animals with amino-nucleoside nephropathy (Eur. J. Clin. Invest. 1998; 28: 877-883; Renal Physiol. 1984; 7: 218-226). Inhibition of lipid peroxidation also has been observed in human nonneoplastic lung tissue (Gen. Pharmacol. 1996; 27: 855-859).

Mopidamole is known to possess antithrombotic and additionally antimetastatic properties.

In WO 01/|30353| is disclosed that fibrin-dependent microcirculation disorders can be treated by dipyridamole, for example microcirculation disorders caused by metabolic diseases, inflammatory reactions or autoimmune diseases, furthermore peripheral microcirculation disorders or microcirculation disorders associated with increased cell fragmentation.

Furthermore, WO 02/|085331| discloses that NO-dependent microcirculation disorders can be treated by dipyridamole, due to the activity as free radical scavenger.

WO 02/|342481| discloses a method for increasing tissue perfusion with blood by co-administration of an agent that increases cGMP synthesis and an agent that inhibits cGMP degradation in the cells of the blood vessel walls or in blood cells, e.g. by co-administration of a statin and dipyridamole.

Matrix metalloproteinases (MMPs) are a family of proteolytic enzymes which degrade the extracelular matrix or components of the basement membrane and participate in various physiologic and pathologic processes. MMP-9, also referred to Gelatinase B, is the main matrix metalloproteinase that cleaves Collagen Type IV. MMP-9 also has significant elastinolytic activity, cleaves aggrecan, a cartilage proteoglycan, and cleaves link protein, a glycoprotein that stabilizes the interaction between aggrecans and hyaluronate in proteoglycan aggregates. MMP-9 is constitutively expressed in trophoblasts, osteoclasts, neutrophils, and macrophages. However, abnormal expression can be induced in a variety of cells exposed to inflammatory stimuli, including monocytes (see Example 1). By locally degrading extracellular matrix components, MMP-9 can enhance leukocyte emigration from the vascular compartment into atherosclerotic tissues or generate chemotactic peptides. Abnormal expression of MMP-9 is thought to contribute to the progressive deterioration of the elastic lamellae characteristic of aneurysm formation, and neutralization of MMP-9 activity suppresses the development of aortic aneurysms.

### BRIEF SUMMARY OF THE INVENTION

It has now surprisingly been found that dipyridamole and mopidamole reduce MMP-9 gene expression thus providing an approach for a method of treatment and/or prevention of MMP-9-dependent disorders.

The finding that dipyridamole and mopidamole downregulate MMP-9 synthesis thus contributing to stabilize cell membranes provides a rationale also for combination treatment together with other antithrombotic agents, such as platelet aggregation inhibitors, e.g. acetylsalicalic acid (ASA), clopidogrel or ticlopidine or the pharmaceutically acceptable salts thereof, fibrinogen receptor antagonists (Abciximab, RDGS-peptides, synthetic i.v. or oral fibrinogen antagonists, e.g. fradafiban, lefradafiban or pharmaceutically acceptable salts thereof), heparin and heparinoids or antithrombins, or for combination treatment using additional cardiovascular therapies such as treatment with ACE inhibitors, Angiotensin II antagonists, Ca-antagonists or lipid-lowering agents such as the statins. It has been reported that statins, independent from their lipid-lowering activity, reduce the expression of MMP-9, providing a rationale for a preferred combination of dipyridamole with a statin in the treatment of MMP-9 dependent disorders (J. Vasc. Surg. 2002, 36(1),: 158-63).

ASA inhibits aggregation through direct effects on the platelet, in more detail, by irreversibly acetylating platelet cyclooxygenase, thus inhibiting the production of thromboxane, which is strongly thrombotic. In high doses, however, aspirin crosses over into endothelial cells (N. Eng. J. Med. 1984; 311: 1206-1211), where it interrupts the production of prostacyclin, a potent natural inhibitor of platelet aggregation and by-product of the “arachidonic cascade” (N. Engl. J. Med. 1979; 300: 1142-1147). These observations led to the concept of low-dose antiplatelet therapy with ASA to maximize inhibition of thromboxane while minimizing the loss of prostacyclin (Lancet 1981; 1: 969-971). In combination with dipyridamole according to the invention also the low-dose ASA concept is preferred.

Viewed from one aspect the present invention provides a method of treatment of the human or non-human animal body, preferably mammalian body, for treating and/or preventing MMP-9-dependent disorders or medical conditions, accompanied or characterized by global elevation of MMP-9 in the plasma or localized elevation of MMP-9 at an inflammatory site, said method comprising administering to said body an effective amount of a pharmaceutical composition comprising an active ingredient selected from dipyridamole, mopidamole and the pharmaceutically acceptable salts thereof, optionally in combination with one or more other antithrombotic agents, ACE inhibitors, Angiotensin II antagonists, Ca-antagonists or lipid-lowering agents.

Viewed from a different aspect the present invention provides the use of an active ingredient selected from dipyridamole, mopidamole and the pharmaceutically acceptable salts thereof, optionally in combination with one or more other antithrombotic agents, ACE inhibitors, Angiotensin II antagonists, Ca-antagonists or lipid-lowering agents, for the manufacture of a pharmaceutical composition for the treatment of the human or non-human animal body, preferably mammalian body, for treating and/or preventing MMP-9-dependent disorders or medical conditions accompanied or characterized by elevated MMP-9 plasma levels,.

### DETAILED DESCRIPTION OF THE INVENTION

The invention provides a new approach for the treatment and/or prevention of MMP-9-dependent disorders or medical conditions accompanied or characterized by elevated MMP-9 plasma levels, said method comprising administering to said body an effective amount of a pharmaceutical composition comprising an active ingredient selected from dipyridamole, mopidamole and the pharmaceutically acceptable salts thereof, optionally in combination with one or more other antithrombotic agents, ACE inhibitors, Angiotensin II antagonists, Ca-antagonists or lipid-lowering agents. MMP-9-dependent disorders are meant to be such disorders or medical conditions being accompanied or characterized by elevated MMP-9 plasma levels or such conditions where elevated MMP-9 plasma levels are involved or contribute in pathogenesis or progression of the disorder. This is the case for instance in disorders wherein sequential inflammatory reactions contribute or lead to development of vascular syndromes, damages or diseases, atherosclerotic damages or arthritic conditions. Elevated MMP-9 plasma levels are reported in connection with several disorders in the scientific literature.

Recently elastic fibers in the vessel walls were found to control smooth muscle cell (SMC) proliferation. In the case of low concentration of elastic fibers or disruption by intervention, the control of SMC proliferation by elastic fibers is lost. In the case of elevated MMP-9 plasma levels, it must be assumed that structural proteins are digested by the metalloproteinases leading to restenosis.

The indication “MMP-9-dependent disorders” should be understood in a non-limiting manner to comprise
(a) vascular syndromes, damages or diseases,
   such as development of arterial aneurysm (Circ. Res. 2001, 89(6), 509-16), aortic aneurysm (J. Vasc. Interv. Radiol. 2000 11(10): 1345-52; J. Clin. Invest. 2002, 110(5): 625-32; Prevention: J. Clin. Invest. 2000, 105(11): 1641-9), left ventricular enlargement after myocardial infarction (J. Clin. Invest. 2000, 106(1): 55-62), formation of either a plaque rupture and subsequent thromboembolic occlusion of a vessel such as in myocardial infarction or stroke or in the form of massive bleeding from an aneurysm which has lost or weakened its structural elements and then ruptures, stenosis or restenosis after balloon angioplasty or implantation of devices in particular stents, valves, filters, intravenous or intra-arterial lines,
(b) atherosclerotic damages,
   such as premature coronary atherosclerosis (Clin. Chem. Lab. 2001, 39(5): 380-4; Arterioscler. Thromb. Vasc. Biol. 2001, 21(9): 1446-50), stabilization of atherosclerotic plaques (Yonsei Med J 2000, 41(1): 82-8), particularly what is understood as plaques with thinned cap or plaques exposed to elevated levels of shear stress known to rupture easily (vulnerable plaque),
(c) arthritic conditions,
   such as psoriatic arthritis, rheumatoid arthritis, osteoarthritis, temporomandibular joint arthritis (Clin. Exp. Rheumatol. 2001, 19(6): 760; Arthritis Rheum. 2001, 44(9): 2024-8, J. Orofac. Pain 2000, 14(1): 20-30; J. Rheumatol. 2001, 28(3): 485-89), lyme arthritis (Arthritis Rheum. 2001, 44(6): 1401-10),
(d) sequential inflammatory reactions that lead to vascular syndromes, damages or diseases, atherosclerotic damages or arthritic conditions,
(e) acute inflammatory reactions,
   such as sepsis, pneumonia, thrombosis and in acute lung injuries,
(f) autoimmune reactions,
   such as lupus erythematosus (Clin. Exp. Immunol. 2002, 127(2): 393-8), rheumatoid synovitis (Rheumatology 2002, 41(1): 78-87),
(g) proliferative diseases,
   such as cancer, e.g. stage IIB osteosarcoma around the knee (J. Bone Joint. Surg. Br. 2002, 84(5): 706-11), cystic renal carcinomas (J. Urol. 2002, 168(1): 19-22), prostate cancer (Acta. Oncol. 2002, 41(3): 289-96), bladder cancer (J. Med. Invest. 2001, 48(1-2): 31-43), non-Hodgkin's lymphoma (Blood 1991, 77(11):2475-81), leukaemia (Br. J. Haematol. 2002, 117(4): 835-41), pancreatic carcinomas with liver metastasis, colon carcinomas with liver metastasis (J. Surg. Oncol. 2002, 80(2): 105-10, colorectal cancer (Br. J. Cancer 2002, 86(12): 1876-83), hepatocellular carcinoma (World J. Gastroenterol. 2002, 8(3): 385-92), head and neck squamous cell carcinoma (Cancer 2002, 94(5): 1483-91), ovarian carcinoma (Int. J. Oncol. 2000, 17(4): 673-81), including tumour invasion, metastasis and angiogenesis (Clin. Cancer Res. 2000 6(12): 4823-30; Pathol. Oncol. Res. 2001, 7(1):14-23),
(h) or, as further indication,
   the risk of thrombolytic/fibrinolytic therapy-induced major bleedings, including intracranial haemorrhages, e.g. in fibrinolytic therapy with tissue plasminogen activators (such as rt-PA or TNK-PA), streptokinase, staphylokinase, urokinase or a derivative thereof, whereby this risk is reduced by the method of the invention.

The method of prevention aspect of the invention applies especially to the indications of groups (a), (b), (c) (d) and (h).

According to the method of treatment and/or prevention according to the invention it is of advantage to maintain a plasma level of dipyridamole or mopidamole of about 0.2 to 5 μmol/L, preferably of about 0.4 to 5 μmol/L, especially of about 0.5 to 2 μmol/L or particularly of about 0.8 to 1.5 μmol/L. This can be achieved using any of the oral dipyridamole retard, instant or the parenteral formulations on the market, the retard formulations being preferred, for instance those available under the trademark Persantin®, or, for the combination therapy with low-dose ASA, using those formulations available under the trademark Asasantin® or Aggrenox®. Dipyridamol retard formulations are also disclosed in EP-A-|0032562|, instant formulations are disclosed in EP-A-|0068191| and combinations of ASA with dipyridamole are disclosed in EP-A-|0257344| which are incorporated by reference. In case of mopidamole also oral retard, instant or a parenteral formulations can be used, e.g. those disclosed in GB 1,051,|218| or EP-A-0,108,|898| which are incorporated by reference, retard formulations being preferred.

Dipyridamole or mopidamole can be administered orally in a daily dosage of 25 to 1000 mg, preferably 50 to 900 mg, more preferred 100 to 480 mg, most preferred 150 to 400 mg. For long-term treatment it is of advantage to administer repeated doses such as a dose of 50 to 500 mg, preferably 50 to 100 mg of dipyridamole or mopidamole retard or any other instant release formulation three or four times a day. For parenteral administration dipyridamole or mopidamole could be given in a dosage of 0.5 to 5 mg/kg body weight, preferably 1 to 3.5 mg/kg body weight, during 24 hours as slow i.v. infusion (not faster than 0.2 mg/min).

As already mentioned hereinbefore dipyridamole, mopidamole or a pharmaceutically acceptable salt thereof can be used alone in a monopreparation or in combination with other antithrombotic agents, ACE inhibitors, Angiotensin II antagonists, Ca-antagonists or lipid-lowering agents for the treatment of MMP-9-dependent disorders.

Furthermore, the method of treatment and/or prevention according to the invention can be combined with any basic method of treatment or prevention known in the art for the above-identified disorders.

In case of atherosclerotic disorders this basic method of treatment or prevention may comprise administration of lipid-lowering agents such as HMG-Co-A reductase inhibitors or statins in the doses known in the art.

In case of arthritic conditions or inflammatory reactions this basic method of treatment or prevention may comprise administration of nonsteroidal anti-inflammatory drugs (NSAIDs) in the doses known in the art. Suitable NSAIDs for combination treatment are meant to include all COX (cyclooxygenase) inhibitors, e.g.

non-selective COX-inhibitors such as acetylsalicyclic acid, mesalazin,

ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen,

indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac,

mefenamic acid, meclofenamic acid, flufenamic acid, nifluminic acid, tolfenamic acid, diflunisal, flufenisal, piroxicam, tenoxicam, lornoxicam and nimesulide and the pharmaceutically acceptable salts thereof,

as well as selective COX 2-inhibitors such as meloxicam, celecoxib and rofecoxib and the pharmaceutically acceptable salts thereof.

In such combinations with any basic method of treatment or prevention known in the art each active ingredient can be administered either in accordance with its usual dosage range or a dose below its usual dosage range. The dosage for the combined NSAIDs or immunsuppressives is appropriately 1/50 of the lowest dose normally recommended up to 1/1 of the normally recommended dosage, preferably 1/20 to ½ and more preferably 1/10 to ⅕. The normally recommended dose for the combined drug should be understood to be the dose disclosed for example in Rote Liste® 2002, Editio Cantor Verlag Aulendorf, Germany, or in Physician's Desk Reference.

In case of autoimmune reactions this basic method of treatment or prevention may comprise administration of immunsuppressives such as cyclosporin A and derivatives thereof, mycophenolatemofetil, FK 506, OKT-3, ATG, 15-desoxyspergualin, mizoribine, misoprostol, rapamycin, reflunomide, azathioprine or NF-Kappa B-inhibitors in the doses known in the art.

In case of proliferative diseases this basic method of treatment or prevention may comprise administration of anti-tumour therapeutic agents, for topoisomerase inhibitors (e.g. etoposide), mitosis inhibitors (e.g. vinblastine), compounds which interact with nucleic acids (e.g. cis-platin, cyclophosphamide, adriamycin), hormone antagonists (e.g. tamoxifen), inhibitors of metabolic processes (e.g. 5-FU etc.), cytokines (e.g. interferons) or antibodies, etc.

In case of reduction of the risk of thrombolytic/fibrinolytic therapy-induced major bleedings the method of treatment and/or prevention according to the invention may combined with administration of activated coagulation factor VII (VIIa) or of a functional derivative thereof as disclosed in WO 02/|49665|.

Dipyridamole or mopidamole in combination with low-dose ASA may be administered orally in a daily dosage of 10 to 30 mg of ASA together with 50 to 1200 mg of dipyridamole or mopidamole, preferably 100 to 1200 mg, more preferred 160 to 960 mg, most preferred 160 to 480 mg of dipyridamole or mopidamole, for instance in a weight ratio between 1 to 5 and 1 to 12, most preferred a weight ratio of 1 to 8, for instance 25 mg of ASA together with 200 mg of dipyridamole or mopidamole, typically given two times a day.

Other antithrombotic compounds would be given at 0.1 to 10 times, preferably at 0.3 to 5.0 times, most preferred at 0.3 to 2.0 times the clinically described dose (e.g. ® 2002; fradafiban, lefradafiban: EP-A-|0483667|), together with a daily dosage of 25 to 900 mg, preferably 50 to 480 mg, most preferred 75 to 400 mg of dipyridamole or mopidamole.

For combination treatment using dipyridamole or mopidamole together with ACE inhibitors any ACE inhibitor known in the art would be suitable, e.g. benazepril, captopril, ceronapril, enalapril, fosinopril, imidapril, lisinopril, moexipril, quinapril, ramipril, trandolapril or perindopril, using the dosages known in the art, for instance as described in Rote Liste® 2002, Editio Cantor Verlag Aulendorf.

For combination treatment using dipyridamole or mopidamole together with Angiotensin II antagonists any Angiotensin II antagonist known in the art would be suitable, e.g. the sartans such as candesartan, eprosartan, irbesartan, losartan, telmisartan, valsartan, olmesartan or tasosartan, using the dosages known in the art, for instance as described in Rote Liste® 2002, Editio Cantor Verlag Aulendorf.

For combination treatment using dipyridamole or mopidamole together with Ca-antagonists any Ca-antagonist known in the art would be suitable, e.g. nifedipine, nitrendipine, nisoldipine, nilvadipine, isradipine, felodipine or lacidipine, using the dosages known in the art, for instance as described in Rote Liste® 2002, Editio Cantor Verlag Aulendorf.

For combination treatment using dipyridamole or mopidamole together with statins any statin known in the art would be suitable, e.g. lovastatin, simvastatin, pravastatin, fluvastatin, atorvastatin or cerivastatin, using the dosages known in the art, for instance as described in Rote Liste® 2002, Editio Cantor Verlag Aulendorf.

With respect to all aspects of the invention mentioned hereinbefore dipyridamole and the salts thereof are preferred.

In order to study the inhibition of MMP-9 gene expression by dipyridamole the following experiment was carried out:

### EXAMPLE 1

Inhibition of MMP-9 Gene Expression in Platelet-Monocyte Aggregates by the Dipyridamole Component of Aggrenox® (AGG)

Aggrenox® (AGG) is a fixed dosed combination of extended-release dipyridamole (DIP) and aspirin (ASA). AGG is recommended in the protection of secondary stroke and transient ischemic attacks. It also increases tissue perfusion in patients with stable angina or Raynaud's disease. It was determined if AGG blocked the synthesis of inflammatory genes produced by platelet-monocyte aggregates.

Human platelets and monocytes were pretreated with Dipyridamole (DIP) (5 μg/ml), ASA (625 ng/ml), or a DIP/ASA mixture (AGG); 5 μg/ml: 625 ng/ml, an 8:1 ratio of DIP/ASA). The cells were adhered to collagen type I. Synthesis of matrix metalloproteinase-9 (MMP-9) was determined. Co-incubation of platelets with monocytes as well as adherence to collagen significantly resulted in a significant increase in MMP-9 synthesis. AGG and DIP reduced MMP-9 expression (53%, 61%, and a 17% reduction in MMP-9 synthesis compared to untreated cells for AGG, DIP, and ASA, respectively; results shown in FIG. 1). The inhibitory actions of AGG on gene expression are due to the DIP component of this |drug|.

### FIGURE LEGEND:

FIG. 1: The Dipyridamole component of Aggrenox attenuates MMP-9 synthesis by monocytes (monos) adherent to platelets (pits) and collagen. Platelets and monocytes were left alone or pretreated with aspirin (ASA: 625 ng/ml), dipyridamole (DIP: 5 μg/ml) or aggrenox (AGG: 8:1 DIP/ASA ratio) for 15 minutes. The cells were subsequently adhered to Collagen Type 1 for 18 hours and MMP-9 expression was measured. The experiments represent the mean±SEM for 9 independent experiments.

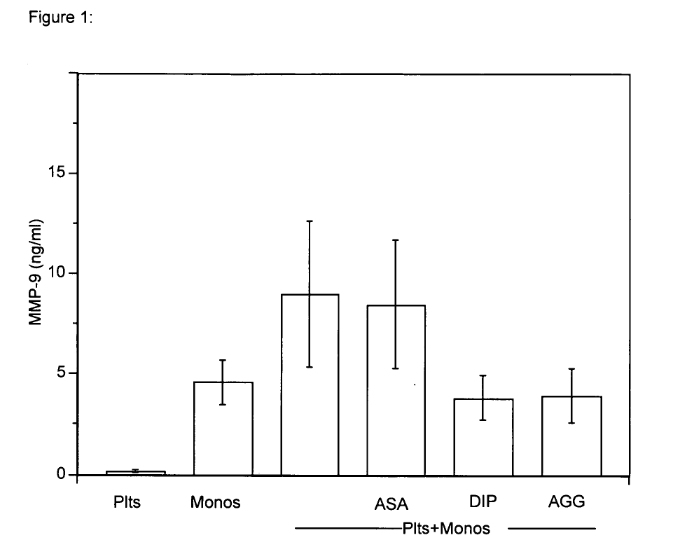

1. A method of treatment of the human or non-human animal body for treating or preventing MMP-9 dependent disorders or of medical conditions, said method comprising administering to said body an effective amount of a pharmaceutical composition comprising an active ingredient selected from dipyridamole, mopidamole and the pharmaceutically acceptable salts thereof, optionally in combination with one or more other antithrombotic agents or optionally in combination with an ACE inhibitor, Angiotensin II antagonist, Ca-antagonist or lipidlowering agent.

2. The method of claim 1, characterized in that the MMP-9 dependent disorder is selected from the group consisting of
(a) vascular syndromes, damages or diseases,
such as development of arterial aneurysm, aortic aneurysm, left ventricular enlargement after myocardial infarction, stenosis or restenosis,
(b) atherosclerotic damages,
such as premature coronary atherosclerosis, stabilization of atherosclerotic plaques,
(c) arthritic conditions,
such as psoriatic arthritis, rheumatoid arthritis, osteoarthritis, temporomandibular joint arthritis, lyme arthritis,
(d) sequential inflammatory reactions that lead to vascular syndromes, damages or diseases, atherosclerotic damages or arthritic conditions,
(e) acute inflammatory reactions,
such as sepsis, pneumonia, thrombosis and in acute lung injuries,
(f) autoimmune reactions,
such as lupus erythematosus,
(g) proliferative diseases,
such as cancer, e.g. stage IIB osteosarcoma around the knee, cystic renal carcinomas, prostate cancer, bladder cancer, non-Hodgkin's lymphoma, leukaemia, pancreatic carcinomas with liver metastasis, colon carcinomas with liver metastasis, colorectal cancer, hepatocellular carcinoma, head and neck squamous cell carcinoma, ovarian carcinoma, including tumour invasion, metastasis and angiogenesis, and
(h) the risk of thrombolytic/fibrinolytic therapy-induced major bleedings, including intracranial haemorrhages, e.g. in fibrinolytic therapy with tissue plasminogen activators (such as rt-PA or TNK-PA), streptokinase, staphylokinase, urokinase or a derivative thereof.

3. The method of claim 2, wherein a plasma level of the active ingredient of about 0.2 to 5 μmol/L is maintained.

4. The method of claim 2, wherein the active ingredient is administered orally in a daily dosage of 25 to 1000 mg or parenterally in a daily dosage of 0.5 to 5 mg/kg body weight.

5. The method of claim 2, wherein the active ingredient is administered alone in a monopreparation.

6. The method of claim 2, wherein the active ingredient is administered in combination with at least one other pharmaceutical active compound selected from the group consisting of an antithrombotic agent, an ACE inhibitor, an Angiotensin II antagonist, a Ca-antagonist and a lipid-lowering agent.

7. The method of claim 2, wherein the MMP-9 dependent disorder is an atherosclerotic disorder and the method comprises administration of a lipid-lowering agent.

8. The method of claim 2, wherein the MMP-9 dependent disorder is an arthritic condition or inflammatory reaction and the method comprises administration of a nonsteroidal anti-inflammatory drug (NSAID).

9. The method of claim 8, wherein the NSAID is selected from the group consisting of meloxicam, celecoxib, rofecoxib and the pharmaceutically acceptable salts thereof.

10. The method of claim 2, wherein the MMP-9 dependent disorder is an autoimmune reaction and the method comprises administration of an immunsuppressive.

11. The method of claim 2, wherein the MMP-9 dependent disorder is a proliferative disease and the method comprises administration of an anti-tumour therapeutic agent.

12. The method of claim 2, wherein the MMP-9 dependent disorder is the risk of thrombolytic/fibrinolytic therapy-induced major bleedings and the method comprises administration of activated coagulation factor VII (VIIa) or of a functional derivative thereof.

13. The method of claim 2, wherein the active ingredient is administered orally in a daily dosage of 50 to 600 mg in combination with 10 to 30 mg of ASA.
